(19) United States
(12) Patent Application Publication
(10) Pub. No.: US 2005/0182078 A1
(43) Pub. Date: Aug. 18, 2005

(54) 2-(Pyridin-3-ylamino)-pyrido[2,3-D]pyrimidin-7-ones

(75) Inventors: Mark R. Barvian, Ann Arbor, MI (US); Peter Laurence Toogood, Ann Arbor, MI (US); Scott Norman VanderWel, Ann Arbor, MI (US)

Correspondence Address: AGOURON PHARMACEUTICALS, INC., 10777 SCIENCE CENTER DRIVE, SAN DIEGO, CA, 92121, US

(73) Assignee: WARNER-LAMBERT COMPANY, LLC

(21) Appl. No.: 11/060,015

(22) Filed: Feb. 16, 2005

Related U.S. Application Data
(60) Provisional application No. 60/545,361, filed on Feb. 18, 2004.

(51) Int. Cl. A61K 31/519; C07D 487/02

(52) U.S. Cl. 514/264.11; 544/279

(57) ABSTRACT

This invention provides compounds of formula I: 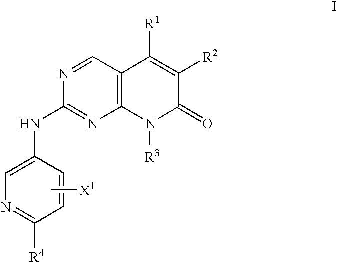
wherein R¹, R², R³, R⁴, and X¹ are as defined in the specification. The 2-(pyridin-3-ylamino)-pyrido[2,3-d]pyrimidin-7-one compounds of formula I, which are inhibitors of cyclin-dependent kinases 2 and 4 (CDK2 and CDK4), are useful in treating cell proliferative disorders.

This application claims the benefit of U. S. Provisional Application No. 60/545,361 filed on Feb. 18, 2004, the contents of which is hereby incorporated by reference in its entirety.

### FIELD OF THE INVENTION

This invention relates to 2-(pyridin-3-ylamino)-pyrido[2,3-d]pyrimidin-7-ones that are potent inhibitors of cyclin-dependent kinases. The compounds of the invention are useful for the treatment of inflammation, and cell proliferative diseases, such as cancer and restenosis.

### BACKGROUND OF THE INVENTION

Cyclin-dependent kinases (“CDK”s) and related serine/threonine protein kinases are important cellular enzymes that perform essential functions in regulating cell division and proliferation. Cyclin-dependent kinase catalytic units are activated by regulatory subunits known as cyclins. At least 16 mammalian cyclins have been identified (Johnson D. G. and Walker C. L., Annu. Rev. Pharmacol. Toxicol. 1999;39:295-312). Cyclin B/CDK1, Cyclin A/CDK2, Cyclin E/CDK2, Cyclin D/CDK4, Cyclin D/CDK6, and probably other heterodimers including CDK3 and CDK7 are important regulators of cell cycle progression. Additional functions of Cyclin/CDK heterodimers include regulation of transcription, DNA repair, differentiation and apoptosis (Morgan D. O., Annu. Rev. Cell. Dev. Biol. 1997; 13261-13291).

Increased activity or temporally abnormal activation of cyclin-dependent kinases has been shown to result in the development of human tumors (Sherr C. J., Science 1996;274:1672-1677). Indeed, human tumor development and other diseases caused by abberant cellular proliferation are commonly associated with alterations in either the CDK proteins themselves or their regulators (Cordon-Cardo C., Am. J. Pathol. 1995;147:545-560; Karp J. E. and Broder S., Nat. Med. 1995;1:309-320; Hall M. et al., Adv. Cancer Res. 1996;68:67-108). For instance, naturally occurring protein inhibitors of CDKs such as p16 and p27 cause growth inhibition in vitro in lung cancer cell lines (Kamb A., Curr. Top. Microbiol. Immunol. 1998;227:139-148).

Small molecule CDK inhibitors may be used in the treatment of cardiovascular disorders such as restenosis and atherosclerosis and other vascular disorders that are due to aberrant cell proliferation. Vascular smooth muscle proliferation and intimal hyperplasia following balloon angioplasty are inhibited by over-expression of the cyclin-dependent kinase inhibitor protein p21 (Chang M. W. et al., J. Clin. Invest., 1995;96:2260; Yang Z-Y. et al., Proc. Natl. Acad. Sci. (USA) 1996;93:9905. Moreover, the purine CDK2 inhibitor CVT-313 (Ki=95 nM) resulted in greater than 80% inhibition of neointima formation in rats (Brooks E. E. et al., J. Biol. Chem. 1997:29207-29211).

CDK inhibitors can be used to treat diseases caused by a variety of infectious agents, including fungi, protozoan parasites such as Plasmodium falciparum, and DNA and RNA viruses. For example, cyclin-dependent kinases are required for viral replication following infection by herpes simplex virus (HSV) (Schang L. M. et al., J. Virol. 1998;72:5626) and CDK homologs are known to play essential roles in yeast.

Selective CDK inhibitors can be used to ameliorate the effects of various autoimmune disorders. Chronic inflammatory disease rheumatoid arthritis is characterized by synovial tissue hyperplasia; inhibition of synovial tissue proliferation should minimize inflammation and prevent joint destruction. Expression of the CDK inhibitor protein p16 in synovial fibroblasts led to growth inhibition (Taniguchi K. et al., Nat. Med. 1999;5:760-767). Similarly, in a rat model of arthritis, joint swelling was substantially inhibited by treatment with a p16 expressing adenovirus. CDK inhibitors may be effective against other disorders of cell proliferation including psoriasis (characterized by keratinocyte hyperproliferation), glomerulonephritis, and lupus.

Certain CDK inhibitors may be useful as chemoprotective agents through their ability to inhibit cell cycle progression of normal untransformed cells (Chen et al. J. Natl. Cancer Institute, 2000;92:1999-2008). Pre-treatment of a cancer patient with a CDK inhibitor prior to the use of cytotoxic agents can reduce the side effects commonly associated with chemotherapy. Normal proliferating tissues are protected from the cytotoxic effects by the action of the selective CDK inhibitor.

### SUMMARY OF THE INVENTION

This invention provides compounds of the formula I: 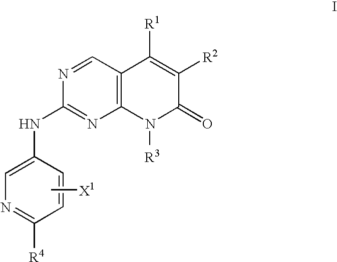
wherein:
X¹ is hydrogen, halogen, C₁-C₆ alkyl, C₁-C₆ haloalkyl, C₁-C₈ alkoxy, C₁-C₈ alkoxyalkyl, CN, NO₂, OR⁵, NR⁵R⁶, CO₂R⁵, COR⁵, S(O)ₙR⁵, CONR⁵R⁶, NR⁵COR⁶, NR⁵SO₂R⁶, SO₂NR⁵R⁶, or P(O)(OR⁵)(OR⁶);
R¹ is hydrogen or C₁-C₃ alkyl;
R² is hydrogen, halogen, C₁-C₆ alkyl, O—C₁-C₆ alkyl, C(O)R⁷, CO₂R⁷, C₁-C₆ alkenyl, C₁-C₆ alkynyl, phenyl, O-phenyl, NR⁷-phenyl, or heteroaryl;
R³ is hydrogen, phenyl, C₁-C₈ alkyl, C₃-C₇ cycloalkyl, or C₃-C₇-heterocyclyl;
R⁴is hydrogen, halogen, C₁-C₈ alkyl, OR⁵, SR⁵, or NR⁵R⁶;
R⁵ and R⁶ are, in each instance independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or
R⁵ and R⁶, when attached to the same nitrogen atom, taken together with the nitrogen to which they are attached, form a heterocyclic ring containing from 3 to 8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂)ₘC(O)NR⁷R⁸, O(CH₂)ₘC(O)OR⁷, and (CH₂)SO₂NR⁷R⁸;
m is 0 to 4;
R⁷ is hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl;
R⁸ and R⁹ are hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl;
and the pharmaceutically acceptable salts, esters, amides, or prodrugs thereof.

Compounds of formula I may contain chiral centers and therefore may exist in different enantiomeric and diastereomeric forms. This invention relates to all optical isomers and all stereoisomers of compounds of the formula I, both as racemic mixtures and as individual enantiomers and diastereoisomers of such compounds, and mixtures thereof, and to all pharmaceutical compositions and methods of treatment defined below that contain or employ them, respectively.

The compounds of formula I and derivatives thereof are selective inhibitors of serine/threonine kinases, cyclin-kinase, dependent kinases 2 and 4 and cyclin-dependent kinase 6. The term derivatives include salts, preferably pharmaceutically acceptable salts, amines, esters and prodrugs of the compounds of formula I. These compounds and derivatives thereof are readily synthesized and can be administered to patients by a variety of methods.

This invention also provides pharmaceutical formulations comprising a therapeutically effective amount of a compound of formula I or a therapeutically acceptable salt thereof and a pharmaceutically acceptable carrier, diluent, or excipient therefor.

The 2-(pyridin-3-ylamino)pyrido[2,3-d]pyrimidinones of formula I and their pharmaceutically acceptable salts and pharmaceutical formulations containing them are useful for treating uncontrolled cell proliferative diseases, including, but not limited to, proliferative diseases such as cancer, restenosis and rheumatoid arthritis. In addition, these compounds and salts thereof are useful for treating inflammation and inflammatory diseases, as anti-infective agents, and as chemoprotective agents.

The above-identified methods of treatment are preferably carried out by administering a therapeutically effective amount of a compound of formula I and pharmaceutically acceptable salts thereof to a subject in need of treatment.

Preferred compounds of the present invention are those having the formula IA: 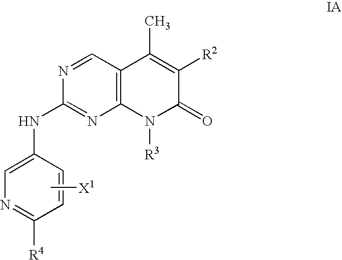
wherein R², R³, R⁴, and X¹ are as defined for formula 1.

In one preferred embodiment of the present invention X¹ is hydrogen.

Preferred embodiments of the present invention include, but are not limited to, the compounds listed below and derivatives, preferably pharmaceutically acceptable salts thereto:
8-isopropyl-2-(pyridin-3-ylamino)-8H-pyrido[2,3-d]pyrimidin-7-one;
8-cyclopentyl-2-(6-methoxy-pyridin-3-ylamino)-8H-pyrido[2,3-d]pyrimidin-7-one;
6-bromo-8-cyclopentyl-2-(6-piperazin-1 -yl-pyridin-3-ylamino)-8H-pyrido[2,3-d]pyrimidin-7-one;
6-bromo-8-cyclopentyl-5-methyl-2-(6-morpholin-4-yl-pyridin-3-ylamino)-8H-pyrido[2,3-d]pyrimidin-7-one;
6-acetyl-8-cyclopentyl-5-methyl-2-(6-morpholin-4-yl-pyridin-3-ylamino)-8H-pyrido[2,3-d]pyrimidin-7-one;
6-bromo-8-cyclopentyl-5-methyl-2-(6-piperazin-1-yl-pyridin-3-ylamino)-8H-pyrido[2,3-d]pyrimidin-7-one;
6-acetyl-8-cyclopentyl-5-methyl-2-(6-piperazin-1-yl-pyridin-3-ylamino)-8H-pyrido[2,3-d]pyrimidin-7-one;
6-ethyl-8-isopropyl-2-(6-piperazin-1-yl-pyridin-3-ylamino)-8H-pyrido[2,3-d]pyrimidin-7-one;
6-benzyl-8-isopropyl-2-(6-piperazin-1-yl-pyridin-3-ylamino)-8H-pyrido[2,3-d]pyrimidin-7-one;
6-acetyl-8-isopropyl-2-(6-piperazin-1-yl-pyridin-3-ylamino)-8H-pyrido[2,3-d]pyrimidin-7-one;
8-isopropyl-7-oxo-2-(6-piperazin-1-yl-pyridin-3-ylamino)-7,8-dihydro-pyrido[2,3-d]pyrimidine-6-carboxylic acid ethyl ester;
6-ethyl-8-(2-methoxy-ethyl)-2-(6-piperazin-1-yl-pyridin-3-ylamino)-8H-pyrido[2,3-d]pyrimidin-7-one;
6-benzyl-8-isopropyl-2-[6-(2-methoxy-ethoxy)-pyridin-3-ylamino]-8H-pyrido[2,3-d]pyrimidin-7-one;
6-acetyl-2-(5-chloro-6-piperazin-1-yl-pyridin-3-ylamino)-8-isopropyl-8H-pyrido[2,3-d]pyrimidin-7-one;
8-isopropyl-2-(6-piperazin-1-yl-pyridin-3-ylamino)-6-thiazol-2-yl-8H-pyrido[2,3-d]pyrimidin-7-one;
3-[6-fluoro-7-oxo-2-(6-piperazin-1-yl-pyridin-3-ylamino)-7H-pyrido[2,3-d]pyrimidin-8-yl]-propionic acid;
8-isopropyl-2-[6-(4-methyl-piperazin-1-yl)-pyridin-3-ylamino]-6-phenoxy-8H-pyrido[2,3-d]pyrimidin-7-one;
6-acetyl-8-cyclopentyl-2-(6-[1,4]diazepan-1-yl-pyridin-3-ylamino)-8H-pyrido[2,3-d]pyrimidin-7-one;
6-ethynyl-8-isopropyl-2-(6-piperazin-1-yl-pyridin-3-ylamino)-8H-pyrido[2,3-d]pyrimidin-7-one;
8-benzyl-2-(6-piperazin-1-yl-pyridin-3-ylamino)-6-vinyl-8H-pyrido[2,3-d]pyrimidin-7-one;
8-(2-cyclopropyl-ethyl)-2-(6-morpholin-4-yl-pyridin-3-ylamino)-6-phenylamino-8H-pyrido[2,3-d]pyrimidin-7-one;
8-cyclopentyl-6-propionyl-2-(3,4,5,6-tetrahydro-2H-[1,2′]bipyridinyl-5′-ylamino)-8H-pyrido[2,3-d]pyrimidin-7-one;
2-[6-(3,5-dimethyl-piperazin-1-yl)-pyridin-3-ylamino]-6-hydroxymethyl-8-isopropyl-8H-pyrido[2,3-d]pyrimidin-7-one;
8-cyclopentyl-6-ethyl-5-methyl-2-(6-piperazin-1-yl-pyridin-3-ylamino)-8H-pyrido[2,3-d]pyrimidin-7-one;
6-chloro-8-isopropyl-5-methyl-2-(6-piperazin-1-yl-pyridin-3-ylamino)-8H-pyrido[2,3-d]pyrimidin-7-one;
6-acetyl-8-isopropyl-5-methyl-2-(6-piperazin-1-yl-pyridin-3-ylamino)-8H-pyrido[2,3-d]pyrimidin-7-one;
8-isopropyl-5-methyl-7-oxo-2-(6-piperazin-1-yl-pyridin-3-ylamino)-7,8-dihydro-pyrido[2,3-d]pyrimidine-6-carboxylic acid ethyl ester;
6-ethyl-8-(2-methoxy-ethyl)-5-methyl-2-(6-piperazin-1-yl-pyridin-3-ylamino)-8H-pyrido[2,3-d]pyrimidin-7-one;
6-benzyl-8-isopropyl-2-[6-(2-methoxy-ethoxy)-pyridin-3-ylamino]-5-methyl-8H-pyrido[2,3-d]pyrimidin-7-one;
6-acetyl-2-(5-chloro-6-piperazin-1-yl-pyridin-3-ylamino)-8-isopropyl-5-methyl-8H-pyrido[2,3-d]pyrimidin-7-one;
8-isopropyl-5-methyl-2-(6-piperazin-1-yl-pyridin-3-ylamino)-6-thiazol-2-yl-8H-pyrido[2,3-d]pyrimidin-7-one;
3-[6-fluoro-5-methyl-7-oxo-2-(6-piperazin-1-yl-pyridin-3-ylamino)-7H-pyrido[2,3-d]pyrimidin-8-yl]-propionic acid;
8-isopropyl-5-methyl-2-[6-(4-methyl-piperazin-1-yl)-pyridin-3-ylamino]-6-phenoxy-8H-pyrido[2,3-d]pyrimidin-7-one;
6-acetyl-8-cyclopentyl-2-(6-[1,4]diazepan-1-yl-pyridin-3-ylamino)-5-methyl-8H-pyrido[2,3-d]pyrimidin-7-one;
8-(2-dimethylamino-ethyl)-6-ethynyl-5-methyl-2-(6-morpholin-4-yl-pyridin-3-ylamino)-8H-pyrido[2,3-d]pyrimidin-7-one;
8-benzyl-5-methyl-2-(6-piperazin-1-yl-pyridin-3-ylamino)-6-vinyl-8H-pyrido[2,3-d]pyrimidin-7-one;
8-(2-cyclopropyl-ethyl)-5-methyl-2-(6-morpholin-4-yl-pyridin-3-ylamino)-6-phenylamino-8H-pyrido[2,3-d]pyrimidin-7-one;
8-cyclopentyl-5-methyl-6-propionyl-2-(3,4,5,6-tetrahydro-2H-[1,2′]bipyridinyl-5′-ylamino)-8H-pyrido[2,3-d]pyrimidin-7-one; and
2-[6-(3,5-dimethyl-piperazin-1-yl)-pyridin-3-ylamino]-6-hydroxymethyl-8-isopropyl-5-methyl-8H-pyrido[2,3-d]pyrimidin-7-one.

### DETAILED DESCRIPTION OF THE INVENTION

This invention comprises compounds of the formula I: 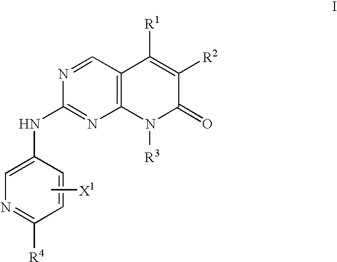
wherein:
X¹ is hydrogen, halogen, C₁-C₆ alkyl, C₁-C₆ haloalkyl, C₁-C₈ alkoxy, C₁-C₈ alkoxyalkyl, CN, NO₂, OR⁵, NR⁵R⁶, CO₂R⁵, COR⁵, S(O)ₙR⁵, CONR⁵R⁶, NR⁵COR⁶, NR⁵COR⁶, SO₂NR⁵R⁶, or P(O)(OR⁵)(OR⁶);
R¹ is hydrogen or C₁-C₃ alkyl;
R² is hydrogen, halogen, C₁-C₆ alkyl, O—C₁-C₆ alkyl, C(O)R⁷, CO₂R⁷, C₁-C₆ alkenyl, C₁-C₆ alkynyl, phenyl, O-phenyl, NR⁷-phenyl, or heteroaryl;
R³ is hydrogen, phenyl, C₁-C₈ alkyl, C₃-C₇ cycloalkyl, or C₃-C₇-heterocyclyl;
R⁴ is hydrogen, halogen, C₁-C₈ alkyl, OR⁵, SR⁵, or NR₅R₆;
R⁵ and R⁶ are, in each instance independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or
R⁵ and R⁶, when attached to the same nitrogen atom, taken together with the nitrogen to which they are attached, form a heterocyclic ring containing from 3 to 8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂)ₘC(O)NR⁷R⁸, O(CH₂)ₘC(O)OR⁷, and (CH₂)SO₂NR⁷R⁸;
m is 0 to 4;
R⁷ is hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl;
R⁸ and R⁹ are hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl;
and the pharmaceutically acceptable salts, esters, amides, or prodrugs thereof.

As the compounds of formula I of this invention may possess asymmetric centers, they are capable of occurring in various stereoisomeric forms or configurations. Hence, the compounds can exist in separated (+)- and (−)-optically active forms, as well as mixtures thereof. The present invention includes all such forms within its scope. Individual isomers can be obtained by known methods, such as optical resolution, optically selective reaction, or chromatographic separation in the preparation of the final product or its intermediate.

The compounds of the present invention can exist in unsolvated forms as well as solvated forms, including hydrated forms. In general, the solvated forms, including hydrated forms, are equivalent to unsolvated forms and are intended to be encompassed within the scope of the present invention.

The present invention also includes isotopically labelled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and esters, amides and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

The compounds of formula I are capable of further forming pharmaceutically acceptable formulations comprising salts, including but not limited to acid addition and/or base salts and solvates of a compound of formula I.

By “alkyl,” in the present invention is meant a straight or branched hydrocarbon radical having from 1 to 10 carbon atoms, preferably 1 to 8 carbon atoms and includes, for example, methyl, ethyl, n-propyl, isopropyl, n-butyl, sec-butyl, isobutyl, tert-butyl, n-pentyl, iso-pentyl, n-hexyl, and the like.

“Alkenyl” means straight and branched hydrocarbon radicals having from 2 to 8 carbon atoms and at least one double bond and includes, but is not limited to, ethenyl, 3-buten-1-yl, 2-ethenylbutyl, 3-hexen-1-yl, and the like. The term “alkenyl” includes, cycloalkenyl, and heteroalkenyl in which 1 to 3 heteroatoms selected from O, S, N or substituted nitrogen may replace carbon atoms.

“Alkynyl” means straight and branched hydrocarbon radicals having from 2 to 8 carbon atoms and at least one triple bond and includes, but is not limited to, ethynyl, 3-butyn-1-yl, propynyl, 2-butyn-1-yl, 3-pentyn-1-yl, and the like.

“Cycloalkyl” means a monocyclic or polycyclic hydrocarbyl group having from 3 to 8 carbon atoms, for instance, cyclopropyl, cycloheptyl, cyclooctyl, cyclodecyl, cyclobutyl, adamantyl, norpinanyl, decalinyl, norbornyl, cyclohexyl, and cyclopentyl. Also included are rings in which 1 to 3 heteroatoms replace carbons. Such groups are termed “heterocyclyl,” which means a cycloalkyl group also bearing at least one heteroatom selected from O, S, N or substituted nitrogen. Examples of such groups include, but are not limited to, oxiranyl, pyrrolidinyl, piperidyl, tetrahydropyran, and morpholine.

By “alkoxy,” is meant straight or branched chain alkyl groups having 1-10 carbon atoms and linked through oxygen. Examples of such groups include, but are not limited to, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, and 3-methylpentoxy. In addition, alkoxy refers to polyethers such as —O—(CH₂)₂—O—CH₃, and the like.

“Acyl” means an alkyl or aryl (Ar) group having from 1-10 carbon atoms bonded through a carbonyl group, i.e., R—C(O)—. For example, acyl includes, but is not limited to, a C₁-C₆ alkanoyl, including substituted alkanoyl, wherein the alkyl portion can be substituted by NR⁸R⁹ or a carboxylic or heterocyclic group. Typical acyl groups include acetyl, benzoyl, and the like.

The alkyl, alkenyl, alkoxy, and alkynyl groups described above are optionally substituted, preferably by 1 to 3 groups selected from NR⁸R⁹, phenyl, substituted phenyl, keto, amino, alkyl, thio C₁-C₆ alkyl, C₁-C₆ alkoxy, hydroxy, carboxy, C₁-C₆ alkoxycarbonyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)ₚphenyl where p is 1, 2, or 3. Perhalo and polyhalo substitution is also included.

Examples of substituted alkyl groups include, but are not limited to, 2-aminoethyl, 2-hydroxyethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, 3-morpholinopropyl, piperazinylmethyl, and 2-(4-methylpiperazinyl)ethyl.

Examples of substituted alkynyl groups include, but are not limited to, 2-methoxyethynyl, 2-ethylsulfanylethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include, but are not limited to, dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The term “anion” means a negatively charged counterion such as chloride, bromide, and trifluoroacetate.

The term “aryl”, as used herein, unless otherwise indicated, includes a C₆- C₁₀ aromatic ring system with no heteroatoms having a single ring (e.g., phenyl), multiple rings (e.g., biphenyl), or multiple fused rings in which at least one is aromatic, (e.g., 1,2,3,4-tetrahydronaphthyl, naphthyl, anthryl, or phenanthryl), wherein each aromatic ring in said aryl ring system can be optionally substituted with from one to three substituents independently selected from halogen, lower alkyl, lower alkoxy, lower alkylthio, trifluoromethyl, lower acyloxy, carbocyclic, heteroaryl, and hydroxy. A preferred aryl is phenyl which can be either unsubstituted or substituted with one, two or three substituents selected from the group consisting of halo, (C₁-C₄)alkyl optionally substituted with from one to three halogen atoms and (C₁-C₄)alkoxy optionally substituted with from one to three halogen atoms.

The term “aryloxy”, as used herein, unless otherwise indicated, means “aryl-O—”, wherein “aryl” is as defined above.

The term “heteroaryl”, as used herein, unless otherwise indicated, includes an aromatic heterocycle containing five to ten ring members, of which from 1 to 4 can be heteroatoms selected, independently, from N, S and O, and which rings can be unsubstituted, monosubstituted or disubstituted with substituents selected, independently, from the group consisting of halo, (C₁-C₄)alkyl, and (C₁-C₄)alkoxy, said alkyl and alkoxy groups being optionally substituted with from one to three halogen atoms. Such heteroaryl groups include, but are not limited to, thienyl, furanyl, thiazolyl, triazolyl, imidazolyl, isoxazolyl, oxadiazolyl, tetrazolyl, pyridyl, pyrrolyl, thiadiazolyl, oxadiazolyl, oxathiadiazolyl, thiatriazolyl, pyrimidinyl, isoquinolinyl, quinolinyl, napthyridinyl, phthalimidyl, benzimidazolyl, and benzoxazolyl. A preferred heteroaryl is pyridine.

The term “heteroaryloxy”, as used herein, unless otherwise indicated, means “heteroaryl-O”, wherein heteroaryl is as defined above.

The term “leaving group”, as used herein, refers to any group (X) that can depart from the carbon to which it is attached carrying with it the two electrons that comprise the bond between the leaving group and that carbon (the X—C bond). Typical leaving groups include but are not limited to: halides (e.g. F⁻, Cl⁻, Br⁻, I⁻), esters, (e.g. acetate), sulfonate esters (e.g. mesylate, tosylate), ethers (EtO⁻, PhO⁻), sulfides (PhS⁻, MeS⁻), sulfoxides, and sulfones.

The term “one or more substituents”, as used herein, refers to a number of substituents that equals from one to the maximum number of substituents possible based on the number of available bonding sites.

By the terms “halo” or “halogen” in the present invention is meant fluorine, bromine, chlorine, and iodine.

The term “cancer” includes, but is not limited to, the following cancers: cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's Disease, hairy cell leukemia, and other leukemias.

The term “treating”, as used herein, refers to reversing, alleviating, inhibiting the progress of, or preventing the disorder or condition to which such term applies, or preventing one or more symptoms of such condition or disorder. The term “treatment”, as used herein, refers to the act of treating, as “treating” is defined immediately above. The term “treating” as used herein may be applied to any suitable mammal. Such mammals include, but are not limited to, canines, felines, bovines, ovines, equines, humans and the like.

This invention further provides compounds of formula I that are useful for treating abnormal cell proliferation such a cancer. The invention provides a method of treating the abnormal cell proliferation disorders such as a cancer selected from the group consisting of cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's, hairy cells, and leukemia, comprising administering a therapeutically effective amount of a compound of formula I, or a pharmaceutically acceptable salt thereof, to a subject in need of such treatment.

A further embodiment of this invention is a method of treating subjects suffering from diseases caused by vascular smooth muscle cell proliferation. Compounds within the scope of the present invention effectively inhibit vascular smooth muscle cell proliferation and migration. The method comprises administering to a subject in need of treatment an amount of a compound of formula I, or a pharmaceutically acceptable salt thereof, sufficient to inhibit vascular smooth muscle proliferation, and/or migration.

This invention further provides a method of treating a subject suffering from gout comprising administering to said subject an amount of a compound of formula I, or a pharmaceutically acceptable salt thereof, sufficient to treat the condition.

This invention further provides a method of treating a subject suffering from kidney disease, such as polycystic kidney disease, comprising administering to said subject in need of treatment an amount of a compound of formula I, or a pharmaceutically acceptable salt thereof, sufficient to treat the condition.

Because of the selective inhibitory activity against CDKs and other kinases, the compounds of the present invention are also useful for studying the mechanism of action of those kinases, both in vitro and in vivo.

Many of the compounds of the present invention are selective inhibitors of cyclin dependent kinases CDK2 and CDK4, which is to say that they inhibit CDK2 and CDK4 more than they inhibit other tyrosine kinases and other serine-threonine kinases. Compounds of the present invention also may inhibit CDK6 at similar concentrations to those necessary for inhibition of CDK4.

A preferred embodiment of the present invention provides a method of inhibiting CDK2 and/or CDK4 comprising administration of a compound of formula I in an amount that selectively inhibits CDK2 and/or CDK4. The term “selectively inhibits” means that the preferred compound inhibits CDK2 and/or CDK4 at a lower dose than is required to inhibit other kinases.

The term “pharmaceutically acceptable salts, esters, amides, or prodrugs” as used herein refers to those salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefittrisk ratio, and effective for their intended use, as well as the zwitterionic forms, where possible, of the compounds of the invention.

The term “salts” refers to the relatively non-toxic, inorganic and organic acid or base addition salts of compounds of the present invention. These salts can be prepared in situ during the final isolation and purification of the compounds or by separately reacting the purified compound in its free base or free acid form with a suitable organic or inorganic acid or base and isolating the salt thus formed. In so far as the compounds of formula I of this invention are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the basic compounds of Formula I are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Such acid addition salts may be prepared from inorganic acids. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like.

Such acid addition salts may also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like.

Pharmaceutically acceptable base addition salts can be formed from acidic compounds of the formula I. Such salts are formed with metals or amines, such as alkali and alkaline earth metals, or organic amines. The base addition salts of acidic compounds of formula I are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents.

Pharmaceutically acceptable base addition salts may include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, N,N-dibenzylethylenediamine, chloroprocaine, choline, diethanolamine, ethylenediamine, N-methylglucamine, and procaine and the like; see, for example, Berge et al., supra. Also contemplated are the salts of amino acids such as arginate, gluconate, galacturonate, and the like. (See, for example, Berge S. M. et al., “Pharmaceutical Salts,” J. Pharm. Sci., 1977;66:1-19 which is incorporated herein by reference.)

Examples of pharmaceutically acceptable, non-toxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. Preferred esters include C₁-C₄ alkyl. Esters of the compounds of the present invention may be prepared according to conventional methods “March's Advanced Organic Chemistry, 5th Edition”. M. B. Smith & J. March, John Wiley & Sons, 2001.

Examples of pharmaceutically acceptable, non-toxic amides of the compounds of this invention include amides derived from ammonia, primary C₁-C₆ alkyl amines and secondary C₁-C₆ dialkyl amines wherein the alkyl groups are straight or branched chain. In the case of secondary amines the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods such as “March's Advanced Organic Chemistry, 5^{th} Edition”. M. B. Smith & J. March, John Wiley & Sons, 2001.

The term “prodrug” refers to compounds that are rapidly transformed in vivo to yield the parent compound of the above formulae, for example, by hydrolysis in blood. A thorough discussion is provided in T. Higuchi and V. Stella, “Pro-drugs as Novel Delivery Systems,” Vol.14 of the A.C.S. Symposium Series, and in Bioreversible Carriers in Drug Design, ed. Edward B. Roche, American Pharmaceutical Association and Pergamon Press, 1987, both of which are hereby incorporated by reference.

An illustration of the preparation of compounds of the present invention is shown in Schemes 1-7 below.

Synthesis

The compounds of the invention may be prepared according to general Scheme 1. The coupling of components A and B generally requires their combination with or without a suitable solvent such as dimethylsulfoxide (DMSO), toluene or acetonitrile, and heating of this mixture to 80-150° C. Both the sulfoxide and the sulfone provide a suitable leaving group or a mixture of the two may be employed. The selection of sulfoxide or sulfone generally depends on the purity of the coupled product obtained, in particular the extent of contamination with 2-hydroxypyrimidine side products.

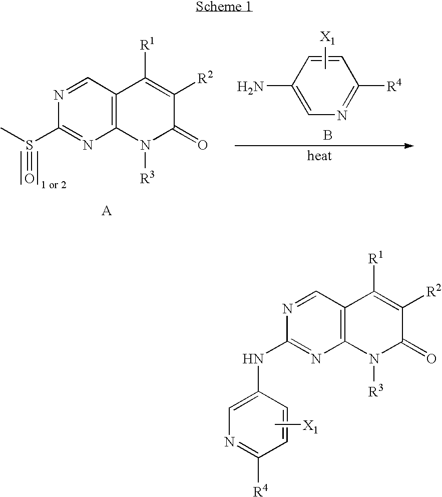

Synthesis of the sulfoxides and sulfones represented by structure A has been described previously in PCT applications WO 98/33798 and WO 01/70741 and WO03/00059. Such intermediates are assembled via established and published protocols (see Barvian et al., J. Med. Chem. 2000, 43, 4606-4616) starting from the commercially available pyrimidine, 4-chloro-2-methylsulfanyl-pyrimidine-5-carboxylic acid ethyl ester. A variety of groups R³ are tolerated by this chemistry and these may be introduced early in the synthetic scheme by displacement of chlorine by an appropriate amine (Scheme 2a), or later by alkylation of the pyridone amide nitrogen (Scheme 2b).

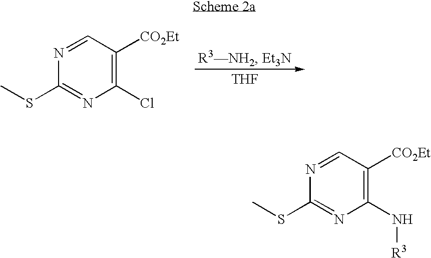

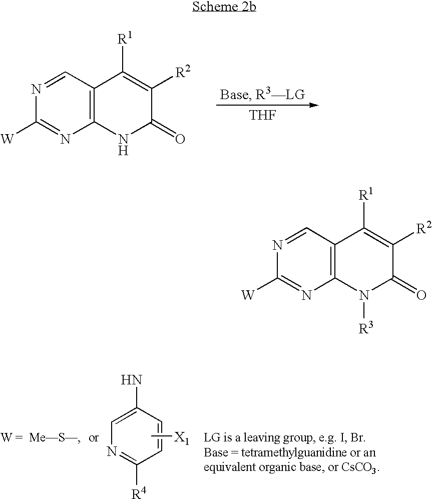

Substituents R² may be introduced using a substituted Horner-Wadsworth Emmons reagent as shown in Scheme 3. Alternatively, further chemistry may be performed at the R² group subsequent to ring closure, including the displacement of fluorine by alkoxides and alkyl amines and anilines.

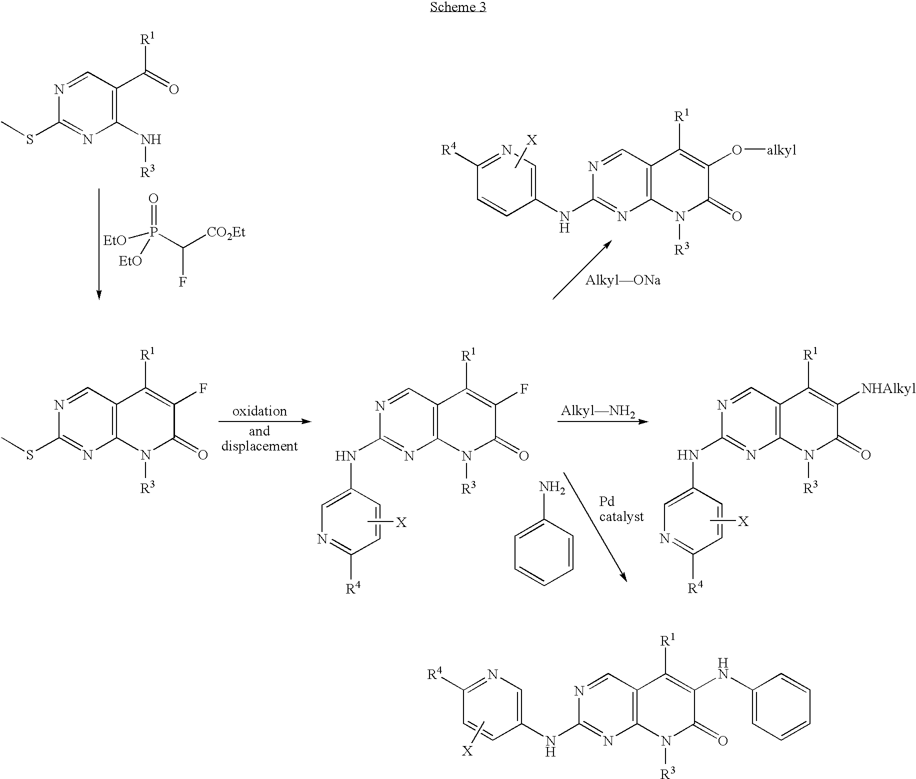

Halogenation at R² may be performed readily using for example N-bromosuccinimide. The halogen can then be replaced using any of a number of reactions known to those of skill in the art, including, but not limited to, metal-halogen exchange, and palladium catalyzed cross-coupling reactions such as the Stille coupling, Suzuki Coupling, carbonylation and related reactions (Scheme 4).

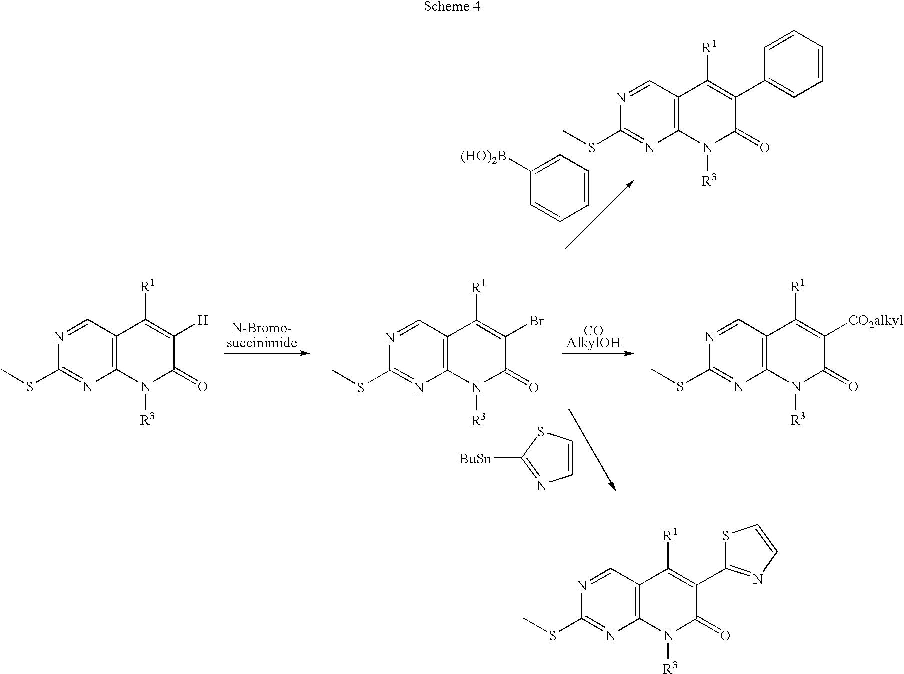

The pyridine derivatives B in Scheme 1, where X¹ is hydrogen can be prepared from commercially available 5-bromo-2-nitropyridine by base or palladium promoted displacement of the bromine by a nucleophile such as an alcohol or a primary or secondary amine, followed by reduction of the nitro group. A representative example of this method is illustrated in Scheme 5. Examples of bases that may be used for this reaction include K₂CO₃, or Na₂CO₃. These bases may be used in the presence of a phase transfer catalyst such as Bu₄NI. Palladium promoted reactions are typically performed with catalysts such as Pd(OAc)₂, Pd₂(dba)₃, or Pd(PPh₃)₄ and the like in nonpolar organic solvents such benzene, toluene, tetrahydrofuran or acetonitrile at temperatures from 25-110° C. These catalysts are typically employed with a suitable ligand, such as 2,2′-(Bis(diphenylphosphino)-1,V-binaphthyl (BINAP), 9,9-Dimethyl-4,5-bis(diphenylphosphino)xanthene (Xantphos), or a related phosphine-based Pd ligand. Reduction of the nitro group is typically performed using Raney Nickel although other reducing agents also may be used including palladium on charcoal, or Fe/HCl. 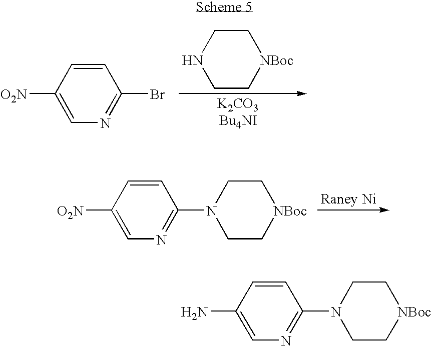
When X¹ is not hydrogen, the pyridine derivatives B are prepared by methods known to those in the art. Examples of representative procedures may be found in Comprehensive Heterocyclic Chemistry, Eds. A. R. Katritzky, C. W. Rees, 1984, Pergamon, N.Y.; Volume 2, Chapter 2.08, Pyridines and their Benzoderivatives: Synthesis, Gurnos Jones. Also, refer to Comprehensive Heterocyclic Chemistry II, Eds. A. R. Katritzky, C. W. Rees., E. Scriven, 1996, Pergamon, N.Y.; Volume 25, Chapter 5.05, Pyridines and their Benzoderivatives: Synthesis, Gurnos Jones. For example, 2,3-dibromo-5-nitropyridine is commercially available and may be substituted selectively at the 2-position to generate side chain fragments B in which X¹═Br (Scheme 6). As described above, a variety of palladium-mediated chemistries are available for subsequent replacement of the bromine by other groups including alkenes, aryls, amines and alcohols and these methods would be well-known to one skilled in organic synthesis.

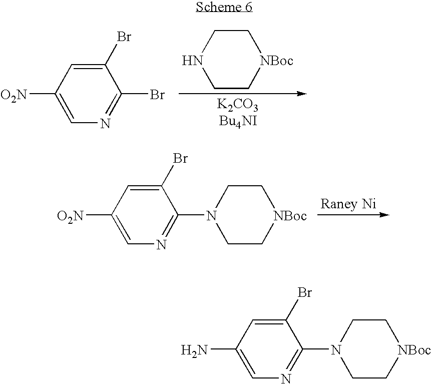

An alternate route to prepare compounds of the present invention involves conversion of the pyridopyrimidine core fragment to a pyridopyrimidine C-2 amine as shown in Scheme 7 and employment of this amine as a nucleophile to displace a leaving group such as bromide or iodide from a pyridine fragment. This reaction proceeds with palladium catalysis to provide the target compounds in equivalent yields to the route shown in Scheme 1. Examples of palladium catalysts that may be employed in this reaction include Pd(OAc)₂, Pd₂(dba)₃, or Pd(PPh₃)₄, and PdCl₂(PPh₃)₂. These catalysts are typically employed with a suitable ligand, such as (2,2′-(Bis(diphenylphosphino)-1,V-binaphthyl) (BINAP), 9,9-Dimethyl-4,5-bis(diphenylphosphino)xanthene (Xantphos) or a related phosphine-based Pd ligand. Typical solvents include dimethoxyethane, tetrahydrofuran, acetonitrile and toluene. Reactions are typically performed at temperatures between 25° C. and 160° C. In some cases, the reaction is accelerated by the presence of electron withdrawing substituents ortho to the leaving group on the pyridine ring (Jonckers, T. H. M. et al., Tetrahedron 2001, 57, 7027-7034).

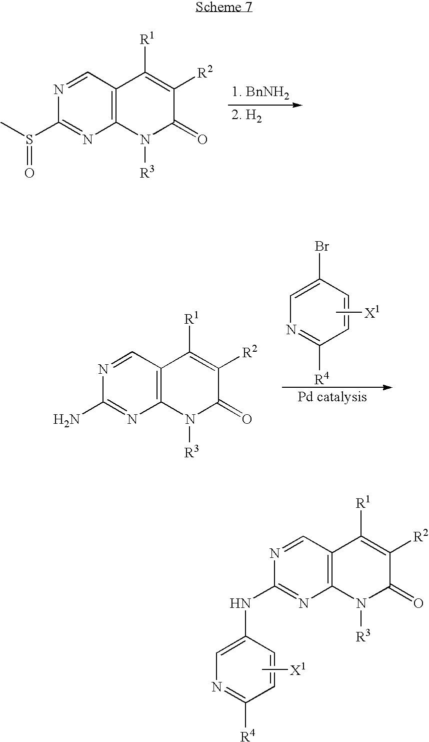

Similar kinds of organometallic couplings maybe performed to install R⁴ late in the synthesis as shown in Scheme 8.

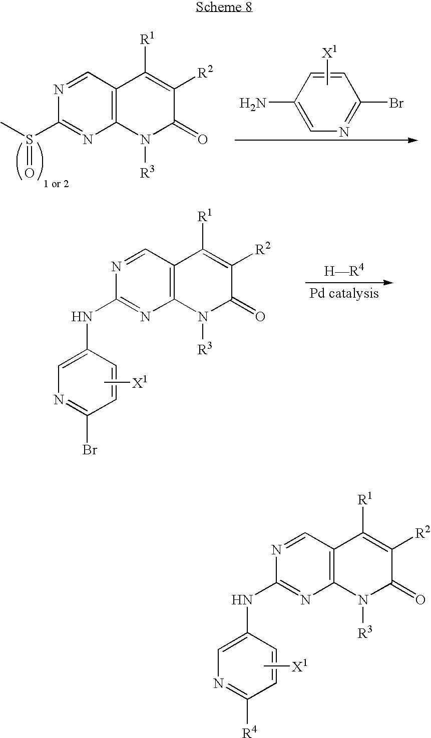

The examples presented below are intended to illustrate particular embodiments of the invention, and are not intended to limit the scope of the specification or the claims in any way.

Those having skill in the art will recognize that the starting materials may be varied and additional steps employed to produce compounds encompassed by the present invention, as demonstrated by the following examples. The following examples are for illustrative purposes only and are not intended, nor should they be construed, as limiting the invention in any manner. Those skilled in the art will appreciate that variations and modifications can be made without violating the spirit or scope of the invention.

### EXAMPLE 1

### 8-Isopropyl-2-(pyridin-3-ylamino)-8H-pyrido[2,3-d]pyrimidin-7-one

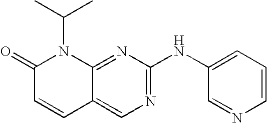

8-Isopropyl-2-(pyridin-3-ylamino)-8H-pyrido[2,3-d]pyrimidin-7-one was prepared according to Scheme 1. mp=156-158° C.

### EXAMPLE 2

### 8-Cyclopentyl-2-(6-methoxy-pyridin-3-ylamino)-8H-pyrido[2,3-d]pyrimidin-7-one

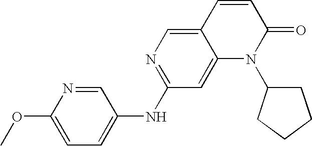

8-Cyclopentyl-2-(6-methoxy-pyridin-3-ylamino)-8H-pyrido[2,3-d]pyrimidin-7-one was prepared according to Scheme 1. mp=181-182° C.

### EXAMPLE 3

### 6-Bromo-8-cyclopentyl-2-(6-piperazin-1-yl-pyridin-3-ylamino)-8H-pyrido[2.3-d]pyrimidin-7-one hydrochloride salt

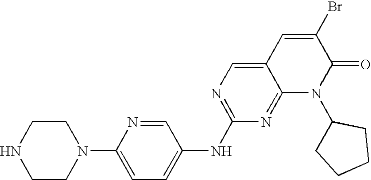

6-Bromo-8-cyclopentyl-2-(6-piperazin-1-yl-pyridin-3-ylamino)-8H-pyrido[2,3-d]pyrimidin-7-one hydrochloride salt was prepared from 4-[5-(6-bromo-8-cyclopentyl-7-oxo-7,8-dihydro-pyrido[2,3-d]pyrimidin-2-ylamino)-pyridin-2-yl]-piperazine-1-carboxylic acid tert-butyl ester according to the general procedure described in Example 6. DMSO (400 MHz, DMSO-d₆) □ 10.09 (s, 1H), 9.22 (s, 2H), 8.73 (s, 1H), 8.45 (s, 1H), 8.44 (s, 1H), 8.33 (s, 1H), 7.98 (d, J=8 Hz, 1H), 7.13 (d, J=9 Hz, 1H), 5.82 (br s, 1H), 3.75 (s, 4H), 3.18 (s, 4H), 2.29-2.12 (m, 2H), 1.87-1.78 (m, 4H), 1.59-1.56 (m, 2H). MS (APCl+) 472.1.

### EXAMPLE 4

### 6-Bromo-8-cyclopentyl-5-methyl-2-(6-morpholin-4-yl-pyridin-3-ylamino)-8H-pyrido[2,3-d]pyrimidin-7-one

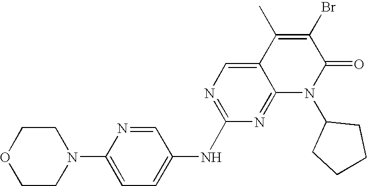

6-Bromo-8-cyclopentyl-5-methyl-2-(6-morpholin-4-yl-pyridin-3-ylamino)-8H-pyrido[2,3-d]pyrimidin-7-one was prepared from 6-bromo-8-cyclopentyl-2-methanesulfinyl-8H-pyrido[2,3-d]pyrimidin-7-one and 6-morpholin-4-yl-pyridin-3-ylamine by the general procedure of Scheme 1. mp=254-256° C. ¹H NMR (400 MHz, CDCl₃) □ ppm 1.61 (m, 2H), 1.81 (m, 2H), 1.96 (m, 2H), 2.24 (m, 2H), 2.57 (s, 3H), 3.48 (m, 4H), 3.83 (m, 4H), 5.90 (m, 1H), 6.67 (d, J=9.0 Hz, 1H), 7.05 (s, 1H), 7.81 (dd, J=9.0, 2.7 Hz, 1H), 8.28 (d, J=2.7 Hz, 1H), 8.68 (s, 1H). m/z 487.1, 485.1 (M+1). CHN C₂₂H₂₅BrN₆O2 0.19 H₂O, Calc. C, 54.06; H, 5.23; N, 17.19; Found C, 53.67; H, 5.11; N, 16.80.

### EXAMPLE 5

### 6-Acetyl-8-cyclopentyl-5-methyl-2-(6-morpholin-4-yl-pyridin-3-ylamino)-8H-pyrido[d]pyrimidin-7-one

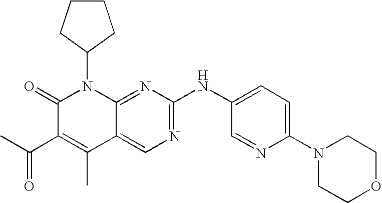

6-Acetyl-8-cyclopentyl-5-methyl-2-(6-morpholin-4-yl-pyridin-3-ylamino)-8H-pyrido[2,3-d]pyrimidin-7-one was prepared from 6-bromo-8-cyclopentyl-5-methyl-2-(6-morpholin-4-yl-pyridin-3-ylamino)-8H-pyrido[2,3-d]pyrimidin-7-one using procedures described in WO 03/062236, incorporated herein by reference. mp=224-226° C.

### EXAMPLE 6

### 6-Bromo-8-cyclopentyl-5-methyl-2-(6-piperazin-1-yl-pyridin-3-ylamino)-8H-pyrido[2,3-d]pyrimidin-7-one

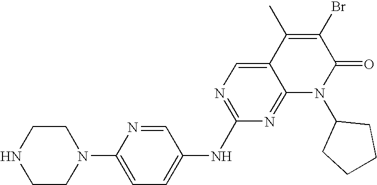

4-[5-(6-Bromo-8-cyclopentyl-5-methyl-7-oxo-7,8-dihydro-pyrido[2,3-d]pyrimidin-2-ylamino)-pyridin-2-yl]-piperazine-1-carboxylic acid tert-butyl ester (1.00 g, 1.71 mmol) was dissolved in EtOAc (20 mL) to which 1.0 N HCl (20 mL) was added and stirred at room temperature overnight. The solvent was then removed in vacuo, suspended in MeCN, then the solid was filtered to give 6-bromo-8-cyclopentyl-5-methyl-2-(6-piperazin-1-yl-pyridin-3-ylamino)-8H-pyrido[2,3-d]pyrimidin-7-one as a yellow solid (0.965 g, 93%). mp=290° C. (foams). ¹H NMR (400 MHz, DMSO-d₆) □ ppm 1.56 (m, 2H), 1.77 (m, 2H), 1.88 (m, 2H), 2.13 (m, 2H), 2.56 (s, 3H), 3.19 (m, 4H), 3.73 (m, 4H), 5.88 (m, 1H), 7.09 (d, J=10.0 Hz, 1H), 7.97 (d, J=7.08 Hz, 1H), 8.44 (d, J=2.4 Hz, 1H), 8.93 (s, 1H), 9.13 (s, 2H), 9.99 (s, 1H). m/z 486.1, 484.1 (M+1). CHN C₂₂H₂₆BrN₇O 3.30 HCl, Calc. C, 43.70; H, 4.88; N, 16.21; Found C, 43.39; H, 5.10; N, 16.14.

### EXAMPLE 7

### 6-Acetyl-8-cyclopentyl-5-methyl-2-(6-piperazin-1-yl-pyridin-3-ylamino)-8H-pyrido[2,3-d]pyrimidin-7-one

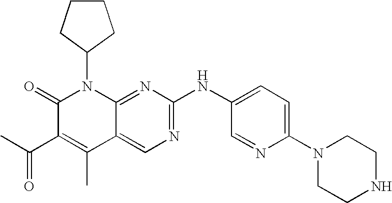

6-Acetyl-8-cyclopentyl-5-methyl-2-(6-piperazin-1-yl-pyridin-3-ylamino)-8H-pyrido[2,3-d]pyrimidin-7-one was prepared from 4-[5-(6-bromb-8-cyclopentyl-7-oxo-7,8-dihydro-pyrido[2,3-d]pyrimidin-2-ylamino)-pyridin-2-yl]-piperazine-1-carboxylic acid tert-butyl ester using procedures described in WO 03/062236. mp=125° C. (foams).

### EXAMPLE 8

### 4-(5-Amino-pyridin-2-yl)-piperazine-1-carboxylic acid tert-butyl ester

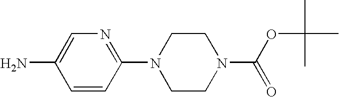

2-Bromo-5-nitro-pyridine (11.39 g, 56.1 mmol), tetrabutylammonium iodide (TBAI) (1.04 g, 0.05 mmol), potassium carbonate (8.53 g, 61.7 mmol) and piperazine-1-carboxylic acid tert-butyl ester (11.5 g, 61.7 mmol) were mixed together in DMSO (100 mL) and gently warmed to 50□ C. for 3 hours and cooled to room temperature overnight. The reaction was diluted with EtOAc (200 mL), the salts were filtered and then the EtOAc was evaporated to leave the DMSO solution. This was diluted with water and a precipitate formed. This precipitate was filtered, washed with water, and then dried in an oven vacuum to give 4-(5-nitro-pyridin-2-yl)-piperazine-1-carboxylic acid tert-butyl ester (16.1 g, 93%) as a light orange solid. ¹H NMR (400 MHz, CDCl₃) □ ppm 1.47 (s, 9H), 3.55 (m, 4H), 3.75 (m, 4H), 6.55 (d, J=9.3 Hz, 1H), 8.21 (dd, J=9.5, 2.7 Hz, 1H), 9.03 (d, J=2.7 Hz, 1H). 4-(5-Nitro-pyridin-2-yl)-piperazine-1-carboxylic acid tert-butyl ester (16.0 g, 51.9 mmol) was dissolved in THF (400 mL), RaNi (4 g) added and placed under a H₂ atmosphere at 50 psi for 5 h. The catalyst was removed by filtration through celite and the solvent evaporated in vacuo to give 4-(5-amino-pyridin-2-yl)-piperazine-1-carboxylic acid tert-butyl ester (14.5 g, 100%). ¹H NMR (400 MHz, CDCl₃) □ ppm 1.46 (s, 9H), 3.31 (m, 6H), 3.53 (m, 4H), 6.56 (d, J=8.8 Hz, 1H), 6.98 (dd, J=8.8, 2.9 Hz, 1H), 7.78 (dd, J=2.9, 0.7 Hz, 1H). m/z 279.1 (M+1).

### EXAMPLE 9

### 6-morpholin-4-yl-pyridin-3-ylamine

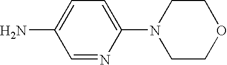

6-morpholin-4-yl-pyridin-3-ylamine was prepared from 2-Bromo-5-nitro-pyridine and morpholine by the general procedure described in Example 8. ¹H NMR (400 MHz, CDCl₃) □ ppm 3.31 (m, 6H), 3.82 (m, 4H), 6.55 (dd, J=8.8, 0.7 Hz, 1H), 6.99 (dd, J=8.8, 2.9 Hz, 1H), 7.79 (dd, J=2.9, 0.5 Hz, 1H). m/z 180.1 (M+1).

Biological Assays

To determine the inhibitory potency and selectivity of compounds of the present invention against CDK4 and related kinases, compounds were evaluated in standard assays routinely used to measure inhibition of cyclin-dependent kinase enzymes and other protein kinases (see for example D. W. Fry et al., J. Biol. Chem. 2001, 276, 16617-16623). The assays were carried out as described below.

Assay for Inhibition of CDK2/Cyclin A

CDK2 enzyme assays for IC₅₀ determinations and kinetic evaluation are performed as follows. 96-well filter plates (Millipore MADVN6550, Bedford, Mass.) are used. The final assay volume is 0.1 mL containing buffer A (20 mM TRIS (tris[hydroxymethyl]aminomethane) (pH 7.4), 50 mM NaCl, 1 mM dithiothreitol, 10 mM MgCl₂), 12 mM ATP containing 0.25 μCi [³²P]ATP, 20 ng CDK2/cyclin A, 1 μg retinoblastoma protein, and the test compound at appropriate dilutions in buffer A (Buffer A alone without added test compound was employed as a control for no inhibition. Buffer A containing excess ethylenediamine tetra acetic acid (EDTA) was used to determine the level of background ³²P in the absence of enzyme activity). All components except the ATP are added to the wells, and the plate is placed on a plate mixer for 2 minutes. The reaction is initiated by addition of [³²P]ATP, and the plate is incubated at 25° C. for 15 minutes. The reaction is terminated by addition of 0.1 mL 20% trichloroacetic acid (TCA). The plate is kept at 4° C. for at least 1 hour to allow the substrate to precipitate. The wells are then washed five times with 0.2 mL 10% TCA, and ³²P incorporation is determined with a beta plate counter (Wallac Inc., Gaithersburg, Md.). The IC₅₀ of the test compound was determined using the median effect method (Chou, T-C and Talalay, P. Applications of the median effect principle for the assessment of low-dose risk of carcinogens and for the quantitation of synergism and antagonism of chemotherapeutic agents. In: New Avenues in Developmental Cancer Chemotherapy (Eds. Harrap, K. T. and Connors, T. A.), pp. 37-64. Academic Press, New York, 1987).

Assay for Inhibition of CDK4/Cyclin D

The CDK4 enzyme assay for IC₅₀ determination and kinetic evaluation is performed as follows. 96-well filter plates (Millipore MADVN6550, Bedford, Mass.) are used. The total volume is 0.1 mL containing buffer A (20 mM TRIS (tris[hydroxymethyl]aminomethane)) (pH 7.4), 50 mM NaCl, 1 mM dithiothreitol, 10 mM MgCl₂), 25 μM ATP containing 0.25 μCi [³²P]ATP, 20 ng CDK4, 1 μg retinoblastoma protein and the test compound at appropriate dilutions in buffer A. Buffer A alone without added test compound was employed as a control for no inhibition. Buffer A containing excess EDTA was used to determine the level of background ³²P in the absence of enzyme activity. All components except the ATP are added to the wells, and the plate is placed on a plate mixer for 2 minutes. The reaction is started by adding [³²P]ATP, and the plate is incubated at 25° C. for 15 minutes. The reaction is terminated by addition of 0.1 mL 20% trichloroacetic acid (TCA). The plate is kept at 4° C. for at least 1 hour to allow the substrate to precipitate. The wells are then washed five times with 0.2 mL 10% TCA, and ³²P incorporation is determined with a beta plate counter (Wallac Inc., Gaithersburg, Md.). The IC₅₀ of the test compound was determined using the median effect method (Chou, T-C and Talalay, P. Applications of the median effect principle for the assessment of low-dose risk of carcinogens and for the quantitation of synergism and antagonism of chemotherapeutic agents. In: New Avenues in Developmental Cancer Chemotherapy (Eds. Harrap, K. T. and Connors, T. A.), pp. 37-64. Academic Press, New York, 1987).

Assay for Inhibition of Fibroblast Growth Factor Receptor Kinase (FGFr)

For FGF receptor (FGFr) tyrosine kinase assays 96-well plates (100 μL/incubation/well), and conditions are optimized to measure the incorporation of ³²P from [γ³²P]ATP into a glutamate-tyrosine co-polymer substrate. Briefly, to each well is added 82.5 μL incubation buffer B (25 mM HEPES (4-(2-hydroxyethyl)-1-piperazinethanesulfonic acid) (pH 7.0), 150 mM NaCl, 0.1% Triton X-100, 0.2 mM PMSF (phenylmethylsulfonylfluoride (protease inhibitor)), 0.2 mM Na₃VO₄, 10 mM MnCl₂) and 750 μg/mL Poly (4:1) glutamate-tyrosine followed by 2.5 μL of the test compound in buffer B and 5 μL of a 7.5 μg/μL FGFr solution to initiate the reaction. Following a 10-minute incubation at 25° C., 10 mL [γ³²P]ATP (0.4 μCi plus 50 μM ATP) is added to each well, and samples are incubated for an additional 10 minutes at 25° C. The reaction is terminated by the addition of 100 μL 30% trichloroacetic acid (TCA) containing 20 mM sodium pyrophosphate and precipitation of material onto glass fiber mats (Wallac). Filters are washed three times with 15% TCA containing 100 mM sodium pyrophosphate, and the radioactivity retained on the filters is counted in a Wallac 1250 Betaplate reader. Nonspecific activity is defined as radioactivity retained on the filters following incubation of samples with buffer alone (no enzyme). Specific enzymatic activity (enzyme plus buffer) is defined as total activity minus nonspecific activity. The concentration of a test compound that inhibited specific activity by 50% (IC₅₀) is determined based on the inhibition curve.

Assay for Inhibition of Plate Derived Growth Factor Receptor (PDGFr)

Enzyme assays for IC₅₀ determinations were performed in 96-well filter plates (Millipore MADVN6550, Bedford, Mass.). The total volume was 100 μL/incubation/well) containing (20 mM Hepes (pH 7.4), 50 μM sodium vanadate, 40 mM magnesium chloride, 10 mM Manganese chloride, 10 μM adenosine triphosphate (ATP) containing [γ³²P]ATP (0.5 μCi, 20 μg of polyglutamic acid/tyrosine (Sigma Chemical Co., St. Louis, Mo.), 10 ng of the intracellular domain of PDGF receptor and appropriate dilutions of the inhibitors. All components except the ATP were added to the well and the plate incubated with shaking for 10 min at 25° C. The reaction is started by adding [γ³²P]ATP, and the plate is incubated for 10 min at 25° C. The reaction is terminated by the addition of 100 μL of 20% trichloroacetic acid (TCA). The plate is kept at 4° C. for at least 15 minutes to allow the substrate to precipitate. The wells were washed 5 times with 0.2 ml of 10% TCA and the radioactivity retained on the filters is counted in a Wallac 1250 Betaplate reader. Nonspecific activity is defined as radioactivity retained on the filters following incubation of samples with buffer alone (no enzyme). Specific enzymatic activity (enzyme plus buffer) is defined as total activity minus nonspecific activity. The concentration of a test compound that inhibited specific activity by 50% (IC₅₀) is determined based on the inhibition curve.

Results from the foregoing assays for compounds of Examples 1 to 7 are presented in Table 1.

TABLE 1

| Compound of | CDK2/A IC₅₀ | CDK4/D | FGFr | PDGFr IC₅₀ |
|---|---|---|---|---|
| EXAMPLE No. | (□M) | IC₅₀ (□M) | IC₅₀ (□M) | (□M) |
| 1 | 0.235 | 0.535 | NA | NA |
| 2 | 0.029 | 0.082 | NA | 29.237 |
| 3 | 0.190 | 0.012 | 0.200 | 0.220 |
| 4 | 1.015 | 0.099 | NA | NA |
| 6 | 3.2 | 0.022 | NA | NA |
| 7 | 0.26 | 0.003 | NA | NA |

(NA = not available)

### Formulations and Administration

The compounds of this invention will typically be formulated with common excipients, diluents, and carriers to provide compositions that are well-suited for convenient administration to mammals. The following examples illustrate typical compositions that are provided in a further embodiment of this invention.

The compounds of the present invention can be formulated and administered in a wide variety of oral and parenteral dosage forms, including transdermal and rectal administration. It will be recognized to those skilled in the art that the following dosage forms may comprise as the active component, either a compound of formula I or a corresponding pharmaceutically acceptable salt or solvate of a compound of formula I.

This invention also comprises a pharmaceutical formulation comprising a therapeutically effective amount of a compound of formula I together with a pharmaceutically acceptable carrier, diluent, or excipient. For preparing pharmaceutical compositions with the compounds of the present invention, pharmaceutically acceptable carriers can be either a solid or liquid. Solid form preparations include powders, tablets, pills, capsules, cachets, suppositories, and dispensable granules. A solid carrier can be one or more substances which may also act as diluents, flavoring agents, binders, preservatives, tablet disintegrating agents, or an encapsulating material.

In powders, the carrier is a finely divided solid such as talc or starch which is in a mixture with the finely divided active component. In tablets, the active component is mixed with the carrier having the necessary binding properties in suitable proportions and compacted in the shape and size desired.

The formulations of this invention preferably contain from about 5% to about 70% or more of the active compound. Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. A preferred form for oral use are capsules, which include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient size molds, allowed to cool, and thereby to solidify.

Liquid form preparations include solutions, suspensions, and emulsions such as water or water/propylene glycol solutions. For parenteral injection, liquid preparations can be formulated in solution in aqueous polyethylene glycol solution, isotonic saline, 5% aqueous glucose, and the like. Aqueous solutions suitable for oral use can be prepared by dissolving the active component in water and adding suitable colorants, flavors, stabilizing and thickening agents as desired. Aqueous suspensions suitable for oral use can be made by dispersing the finely divided active component in water and mixing with a viscous material, such as natural or synthetic gums, resins, methylcellulose, sodium carboxymethylcellulose, or other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors,.stabilizers, buffers, artificial and natural sweeteners, dispersants, thickeners, solubilizing agents, and the like. Waxes, polymers, microparticles, and the like can be utilized to prepare sustained-release dosage forms. Also, osmotic pumps can be employed to deliver the active compound uniformly over a prolonged period.

The pharmaceutical preparations of the invention are preferably prepared in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds of the present invention may be freeze-dried, spray-dried, or evaporatively dried to provide a solid plug, powder, or film of crystalline or amorphous material. Microwave or radio frequency drying may be used for this purpose.

The therapeutically effective dose of a compound of formula I will vary from approximately 0.01 mg/kg to approximately 100 mg/kg of body weight per day. Typical adult doses will be approximately 0.1 mg to approximately 3000 mg per day depending, of course, on the mode of administration, the particular application and the potency of the active component. For example, oral administration may require a total daily dose of from 10 mg to 3000 mg, while an intravenous dose may only require from 0.1 mg to 1000 mg/kg of body weight. These dosages are based on an average human subject having a weight of about 65 to 70 kg. The physician will readily be able to determine doses for subjects whose weight falls outside this range, such as infants and the elderly. The composition can, if desired, also contain other compatible therapeutic agents. The total daily dose may be administered in single or divided doses. Such treatment may be repeated at successive intervals for as long as necessary.

The compounds of the invention may be administered alone or in combination with other drugs and will generally be administered as a formulation in association with one or more pharmaceutically acceptable excipients. The term “excipient” is used herein to describe any ingredient other than the compound of the invention. The choice of excipient will to a large extent depend on the particular mode of administration.

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

### Tablet Formulation of the Compound of Example 7

### Tablet Formulation

| Ingredient | Amount |
|---|---|
| Compound of Example 7 | 50 mg* |
| Lactose | 80 mg |
| Cornstarch (for mix) | 10 mg |
| Cornstarch (for paste) | 8 mg |
| Magnesium Stearate (1%) | 2 mg |
| | 150 mg |

*Quantity adjusted in accordance with drug activity.

A compound of the present invention is mixed with the lactose and cornstarch (for mix) and blended to uniformity to a powder. The cornstarch (for paste) is suspended in 6 mL of water and heated with stirring to form a paste. The paste is added to the mixed powder, and the mixture is granulated. The wet granules are passed through a No. 8 hard screen and dried at 50° C. The mixture is lubricated with 1% magnesium stearate and compressed into a tablet. The tablets are administered to-a patient at the rate of 1 to 4 each day for prevention and treatment of cancer.

Another composition of a typical tablet in accordance with the invention may comprise:

| Ingredient | % w/w |
|---|---|
| Compound of Example 7 | 10.00* |
| Microcrystalline cellulose | 64.12 |
| Lactose | 21.38 |
| Croscarmellose sodium | 3.00 |
| Magnesium stearate | 1.50 |

*Quantity adjusted in accordance with drug activity.

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al., Pharmaceutical Technology On-line, 25(2), 1-14 (2001). Other modified release formulations are described in U.S. Pat. No. 6,106,864.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art. The solubility of compounds of formula (I) used in the preparation of parenteral solutions may be increased by suitable processing, for example, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled dual-, targeted and programmed release.

To a solution of 700 mL of propylene glycol and 200 mL of water for injection is added 20.0 g of the compound of Example 7 of the present invention. The mixture is stirred and the pH is adjusted to 5.5 with hydrochloric acid. The volume is adjusted to 1000 mL with water for injection. The solution is sterilized, filled into 5.0 mL ampoules, each containing 2.0 mL (40 mg of compound), and sealed under nitrogen. The solution is administered by injection to a patient suffering from cancer and in need of treatment.

The compounds of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, J Pharm Sci, 88 (10), 955-958 by Finnin and Morgan (October 1999).

Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus compounds of the invention may be formulated in a more solid form for administration as an implanted depot providing long-term release of the active compound.

The compounds of the invention can also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

A suitable solution formulation for use in an atomiser using electrohydrodynamics to produce a fine mist may contain from 1 μg to 10 mg of the compound of the invention per actuation and the actuation volume may vary from 1 μl to 100 μl. A typical formulation may comprise a compound of this invention, propylene glycol, sterile water, ethanol and sodium chloride. Alternative solvents that may be used instead of propylene glycol include glycerol and polyethylene glycol.

Capsules, blisters and cartridges (made, for example, from gelatin or hydroxypropylmethylcellulose (HPMC)) for use in an inhaler or insufflator may be formulated to contain a powder mix of the compound of the invention, a suitable powder base such as lactose or starch and a performance modifier such as I-leucine, mannitol, or magnesium stearate.

In the case of dry powder inhalers and aerosols, the dosage unit is determined by means of a valve that delivers a metered amount. Units in accordance with the invention are typically arranged to administer a metered dose or “puff” appropriate for the disease state, age and size of the individual. The overall daily dose may be administered in a single dose or, more usually, as divided doses throughout the day.

Formulations for inhaled/intranasal administration may be formulated to be immediate and/or modified release. Modified release formulations include delayed-, sustained-, pulsed-, controlled dual-, targeted and programmed release.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled dual-, targeted and programmed release.

The compounds of the invention may also be administered directly to the eye or ear, typically in the form of drops of a micronised suspension or solution in isotonic, pH-adjusted, sterile saline. Other formulations suitable for ocular and andial administration include ointments, biodegradable (e.g., absorbable gel sponges, collagen) and non-biodegradable (e.g., silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose,or a heteropolysaccharide polymer, for example, gelan gum, may be incorporated together with a preservative, such as benzalkonium chloride. Such formulations may also be delivered by iontophoresis.

Formulations for ocular/andial administration may be formulated to be immediate and/or modified release. Modified release formulations include delayed-, sustained-, pulsed-, controlled dual-, targeted, or programmed release.

The compounds of the invention may be combined with soluble macromolecular entities such as cyclodextrin or polyethylene glycol-containing polymers to improve their solubility, dissolution rate, taste-masking, bioavailability and/or stability.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

This invention provides a pharmaceutical composition for treating a disorder or condition selected from the group consisting of cell proliferative disorders, such as cancer, vascular smooth muscle proliferation associated with atherosclerosis, postsurgical vascular stenosis, restenosis, and endometriosis; infections, including viral infections such as DNA viruses like herpes and RNA viruses like HIV, and fungal infections; autoimmune diseases such as psoriasis, inflammation like rheumatoid arthritis, lupus, type 1 diabetes, diabetic nephropathy, multiple sclerosis, and glomerulonephritis, organ transplant rejection, including host versus graft disease.

The invention and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the spirit or scope of the present invention as set forth in the description and the claims. All documents including patents and published patent applications are incorporated by reference. To particularly point out and distinctly claim the subject matter regarded as invention, the following claims conclude this specification.

1. A compound of formula l: 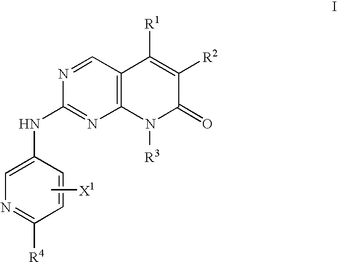
wherein:
X¹ is hydrogen, halogen, C₁-C₆ alkyl, C₁-C₆ haloalkyl, C₁-C₈ alkoxy, C₁-C₈ alkoxyalkyl, CN, NO₂, OR⁵, NR⁵R⁶, CO₂R⁵, COR⁵, S(O)ₙR⁵, CONR⁵R⁶, NR⁵COR⁶, NR⁵SO₂R⁶, SO₂NR⁵R⁶, or P(O)(OR⁵)(OR⁶);
R¹ is hydrogen or C₁-C₃ alkyl;
R² is hydrogen, halogen, C₁-C₆ alkyl, O—C₁-C₆ alkyl, C(O)R⁷, CO₂R⁷, C₁-C₆ alkenyl, C₁-C₆ alkynyl, phenyl, O-phenyl, NR⁷-phenyl, or heteroaryl;
R³ is hydrogen, phenyl, C₁-C₈ alkyl, C₃-C₇ cycloalkyl, or C₃-C₇-heterocyclyl;
R⁴ is hydrogen, halogen, C₁-C₈ alkyl, OR⁵, SR⁵, or NR⁵R⁶;
R⁵ and R⁶ are, in each instance independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or
R⁵ and R⁶, when attached to the same nitrogen atom, taken together with the nitrogen to which they are attached, form a heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, I C₁-C₆ alkyl, C₁-C₆ alkoxy, alkoxycarbonyl, C₁-C₆ alkylcarbonyl, C₁-C₆ alkylcarbonylamino, C₁-C₆ aminoalkyl, C₁-C₆ aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂)ₘC(O)NR⁷R⁸, O(CH₂)ₘC(O)OR⁷, and (CH₂)SO₂NR⁷R⁸;
m is 0 to 4;
R⁷ is hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl;
R⁸ and R⁹ are hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl;
and the pharmaceutically acceptable salts, esters, amides, or prodrugs thereof.

2. A compound according to claim 1 wherein R¹ is methyl.

3. A compound according to claim 1 wherein X¹ is hydrogen.

4. A compound selected from
8-isopropyl-2-(pyridin-3-ylamino)-8H-pyrido[2,3-d]pyrimidin-7-one;
8-cyclopentyl-2-(6-methoxy-pyridin-3-ylamino)-8H-pyrido[2,3-d]pyrimidin-7-one;
6-bromo-8-cyclopentyl-2-(6-piperazin-1-yl-pyridin-3-ylamino)-8H-pyrido[2,3-d]pyrimidin-7-one;
6-bromo-8-cyclopentyl-5-methyl-2-(6-morpholin-4-yl-pyridin-3-ylamino)-8H-pyrido[2,3-d]pyrimidin-7-one;
6-acetyl-8-cyclopentyl-5-methyl-2-(6-morpholin-4-yl-pyridin-3-ylamino)-8H-pyrido[2,3-d]pyrimidin-7-one;
6-bromo-8-cyclopentyl-5-methyl-2-(6-piperazin-1-yl-pyridin-3-ylamino)-8H-pyrido[2,3-d]pyrimidin-7-one;
6-acetyl-8-cyclopentyl-5-methyl-2-(6-piperazin-1-yl-pyridin-3-ylamino)-8H-pyrido[2,3-d]pyrimidin-7-one;
6-ethyl-8-isopropyl-2-(6-piperazin-1-yl-pyridin-3-ylamino)-8H-pyrido[2,3-d]pyrimidin-7-one;
6-benzyl-8-isopropyl-2-(6-piperazin-1-yl-pyridin-3-ylamino)-8H-pyrido[2,3-d]pyrimidin-7-one;
6-acetyl-8-isopropyl-2-(6-piperazin-1-yl-pyridin-3-ylamino)-8H-pyrido[2,3-d]pyrimidin-7-one;
8-isopropyl-7-oxo-2-(6-piperazin-1-yl-pyridin-3-ylamino)-7,8-dihydro-pyrido[2,3-d]pyrimidine-6-carboxylic acid ethyl ester;
6-ethyl-8-(2-methoxy-ethyl)-2-(6-piperazin-1-yl-pyridin-3-ylamino)-8H-pyrido[2,3-d]pyrimidin-7-one;
6-benzyl-8-isopropyl-2-[6-(2-methoxy-ethoxy)-pyridin-3-ylamino]-8H-pyrido[2,3-d]pyrimidin-7-one;
6-acetyl-2-(5-chloro-6-piperazin-1-yl-pyridin-3-ylamino)-8-isopropyl-8H-pyrido[2,3-d]pyrimidin-7-one;
8-isopropyl-2-(6-piperazin-1-yl-pyridin-3-ylamino)-6-thiazol-2-yl-8H-pyrido[2,3-d]pyrimidin-7-one;
3-[6-fluoro-7-oxo-2-(6-piperazin-1-yl-pyridin-3-ylamino)-7H-pyrido[2,3-d]pyrimidin-8-yl]-propionic acid;
8-isopropyl-2-[6-(4-methyl-piperazin-1-yl)-pyridin-3-ylamino]-6-phenoxy-8H-pyrido[2,3-d]pyrimidin-7-one;
6-acetyl-8-cyclopentyl-2-(6-[1,4]diazepan-1-yl-pyridin-3-ylamino)-8H-pyrido[2,3-d]pyrimidin-7-one;
6-ethynyl-8-isopropyl-2-(6-piperazin-1-yl-pyridin-3-ylamino)-8H-pyrido[2,3-d]pyrimidin-7-one; or
8-benzyl-2-(6-piperazin-1-yl-pyridin-3-ylamino)-6-vinyl-8H-pyrido[2,3-d]pyrimidin-7-one.

5. A compound selected from
8-(2-cyclopropyl-ethyl)-2-(6-morpholin-4-yl-pyridin-3-ylamino)-6-phenylamino-8H-pyrido[2,3-d]pyrimidin-7-one;
8-cyclopentyl-6-propionyl-2-(3,4,5,6-tetrahydro-2H-[1,2′]bipyridinyl-5′-ylamino)-8H-pyrido[2,3-d]pyrimidin-7-one;
2-[6-(3,5-dimethyl-piperazin-1-yl)-pyridin-3-ylamino]-6-hydroxymethyl-8-isopropyl-8H-pyrido[2,3-d]pyrimidin-7-one;
8-cyclopentyl-6-ethyl-5-methyl-2-(6-piperazin-1-yl-pyridin-3-ylamino)-8H-pyrido[2,3-d]pyrimidin-7-one;
6-chloro-8-isopropyl-5-methyl-2-(6-piperazin-1-yl-pyridin-3-ylamino)-8H-pyrido[2,3-d]pyrimidin-7-one;
6-acetyl-8-isopropyl-5-methyl-2-(6-piperazin-1-yl-pyridin-3-ylamino)-8H-pyrido[2,3-d]pyrimidin-7-one;
8-isopropyl-5-methyl-7-oxo-2-(6-piperazin-1-yl-pyridin-3-ylamino)-7,8-dihydro-pyrido[2,3-d]pyrimidine-6-carboxylic acid ethyl ester;
6-ethyl-8-(2-methoxy-ethyl)-5-methyl-2-(6-piperazin-1-yl-pyridin-3-ylamino)-8H-pyrido[2,3-d]pyrimidin-7-one;
6-benzyl-8-isopropyl-2-[6-(2-methoxy-ethoxy)-pyridin-3-ylamino]-5-methyl-8H-pyrido[2,3-d]pyrimidin-7-one;
6-acetyl-2-(5-chloro-6-piperazin-1-yl-pyridin-3-ylamino)-8-isopropyl-5-methyl-8H-pyrido[2,3-d]pyrimidin-7-one;
8-isopropyl-5-methyl-2-(6-piperazin-1-yl-pyridin-3-ylamino)-6-thiazol-2-yl-8H-pyrido[2,3-d]pyrimidin-7-one;
3-[6-fluoro-5-methyl-7-oxo-2-(6-piperazin-1-yl-pyridin-3-ylamino)-7H-pyrido[2,3-d]pyrimidin-8-yl]-propionic acid;
8-isopropyl-5-methyl-2-[6-(4-methyl-piperazin-1-yl)-pyridin-3-ylamino]-6-phenoxy-8H-pyrido[2,3-d]pyrimidin-7-one;
6-acetyl-8-cyclopentyl-2-(6-[1,4]diazepan-1-yl-pyridin-3-ylamino)-5-methyl-8H-pyrido[2,3-d]pyrimidin-7-one;
8-(2-dimethylamino-ethyl)-6-ethynyl-5-methyl-2-(6-morpholin-4-yl-pyridin-3-ylamino)-8H-pyrido[2,3-d]pyrimidin-7-one;
8-benzyl-5-methyl-2-(6-piperazin-1-yl-pyridin-3-ylamino)-6-vinyl-8H-pyrido[2,3-d]pyrimidin-7-one;
8-(2-cyclopropyl-ethyl)-5-methyl-2-(6-morpholin-4-yl-pyridin-3-ylamino)-6-phenylamino-8H-pyrido[2,3-d]pyrimidin-7-one;
8-cyclopentyl-5-methyl-6-propionyl-2-(3,4,5,6-tetrahydro-2H-[1,2′]bipyridinyl-5′-ylamino)-8H-pyrido[2,3-d]pyrimidin-7-one; or
2-[6-(3,5-dimethyl-piperazin-1-yl)-pyridin-3-ylamino]-6-hydroxymethyl-8-isopropyl-5-methyl-8H-pyrido[2,3-d]pyrimidin-7-one;
or pharmaceutically acceptable salts thereof.

6. A method of treating a disorder or condition caused by abnormal cell proliferation in a mammal the method comprising administering to said mammal an amount of a compound according to claim 1 that is effective in treating such condition or disorder.

7. The method of claim 6 wherein the disorder or condition being treated is vascular smooth muscle proliferation associated with atherosclerosis; postsurgical vascular stenosis and restenosis; or endometriosis.

8. The method of claim 6 wherein the abnormal cell proliferation is a cancer selected from the group consisting of cancer of the breast, ovary, cervix, prostate, testis, esophagus, stomach, skin, lung, bone, colon, pancreas, thyroid, biliary passages, buccal cavity and pharynx, lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenocarcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's, hairy cells, and leukemia.
